(19) United States
(12) Patent Application Publication
(10) Pub. No.: US 2019/0064102 A1
(43) Pub. Date: Feb. 28, 2019

(54) SYSTEMS AND METHODS FOR THE DETECTION OF GLYPICAN-1

(71) Applicant: CASE WESTERN RESERVE UNIVERSITY, Cleveland, OH (US)

(72) Inventors: Chung Chiun Liu, Cleveland Heights, OH (US); Yifan Dai, Cleveland, OH (US)

(21) Appl. No.: 16/118,216

(22) Filed: Aug. 30, 2018

Related U.S. Application Data
(60) Provisional application No. 62/552,821, filed on Aug. 31, 2017.

(51) Int. Cl. G01N 27/327; G01N 33/68; G01N 33/543; C12Q 1/00

(52) U.S. Cl. CPC G01N 27/3275; C12Q 1/005; G01N 33/54353; G01N 33/6893

(57) ABSTRACT

A sensor for the detection of GPC-1 in a sample includes a substrate, a working electrode and counter electrode formed on a surface of the substrate, and an anti-GPC-1 antibody functionalized or chemically functionalized to a surface of an exposed portion of the working electrode.

### RELATED APPLICATION

This application claims priority from U.S. Provisional Application No. 62/552,821, filed Aug. 31, 2017, the subject matter of which is incorporated herein by reference in its entirety.

### BACKGROUND

Glypican-1 (GPC-1) is a biomarker for early detection of pancreatic cancer. Scientifically, there are reservations about whether GPC-1 could be an early biomarker of pancreatic cancer. The rational for this reservation was based on observations that 70-89% of the patients studied were at an advanced stage—IIb, III and IV stages of pancreatic cancer and only five intraductal papillary mucinous neoplasm (IPMN), a precursor of pancreatic cancer was included in the study. IPMN lesions do not always evolve in malignant tumors.

While the scientific and clinical discussion of the nature of the GPC-1 related to the stage of the pancreatic cancer continue, nevertheless, many studies showed that GPC-1 is crucial for efficient cancer cell growth, metastasis and in the pathogenesis of other diseases. These other diseases can include Alzheimer's disease, prion disease and others. In addition to being a biomarker for pancreatic cancer, GPC-1 can also be considered a biomarker for prostate cancer, colorectal cancer, breast cancer, glioma and others. Therefore, the detection of GPC-1 can be meaningful in the assessment and the observation of the progression of cancer and neurodegenerative disorders in general.

### SUMMARY

Embodiments described herein relate to a detection system, method, and in vitro assay for detecting, identifying, quantifying, and/or determining the levels of Glypican-1 (GPC-1) in a bodily sample as well as to a detection system, method, and in vitro assay for diagnosing, identifying, staging, and/or monitoring cancer or a neurodegenerative disorder in a subject having or suspected of having cancer or a neurodegenerative disorder.

In some embodiments, the system and/or method for detecting, indentifying, quantifying, and/or determining cancer or a neurodegenerative disorders can detect, indentify, quantify, and/or determine the amount or level of GPC-1 in a sample. The system can include an electrochemical biosensor, for detecting, identifying, quantifying, and/or determining the amount or level of GPC-1 in a sample, such as blood. The system and method described herein can provide a single use, disposable, and cost-effective means for simple assessment of GPC-1 in biological samples obtained by non-invasive or minimally invasive means.

In some embodiments, the system and methods described herein includes an electrochemical biosensor, a redox solution, and a measuring device. The electrochemical biosensor can produce a signal that is related to the presence or quantity of the GPC-1 being detected in a sample. In some embodiments, the system can be used to detect and/or quantify GPC-1 that is present in blood or a biological fluid.

In some embodiments, the electrochemical biosensor includes a substrate, a working electrode formed on a surface of the substrate and a counter electrode formed on the surface of the substrate. A dielectric layer covers a portion of the working electrode and counter electrode and defines an aperture exposing other portions of the working electrode and counter electrode. An anti-GPC-1 antibody can be functionalized or chemically functionalized to a surface of an exposed portion of the working electrode. The anti-GPC-1 antibody selectively binds to GPC-1 in a sample, and the GPC-1 once bound is detectable by measuring the current flow between the working electrode and counter electrode.

The redox solution is applied to the working electrode for determining the quantity of GPC-1 in the sample bound to the anti-GPC-1 antibody. The measuring device applies voltage potentials to the working electrode and counter electrode and measures the current flow between the working electrode and counter electrode to determine the level of the GPC-1 in a sample, such as blood.

In some embodiments, the working electrode and the counter electrode include metalized films. The metalized films used to form the working electrode and the counter electrode can independently comprise gold, platinum, palladium, silver, carbon, alloys thereof, and composites thereof. The metalized films can be provided on the surface of the substrate by sputtering or coating the films on the surface and then laser ablating the films to form the working electrode and counter electrode.

In other embodiments, the sensor can include a reference electrode on the surface of the substrate. The dielectric can cover a portion of the reference electrode.

In other embodiments, the anti-GPC-1 antibody can be chemically functionalized to the surface of the working electrode coated with a 3-mercaptopropionic acid (MPA) monolayer.

### BRIEF DESCRIPTION OF THE DRAWINGS

FIG. 1 is a schematic illustration of a biosensor in accordance with an aspect of the application.

FIG. 2 illustrates an image showing the structure and dimensions of this biosensor.

FIGS. 3(A-B) illustrate: (A) a plot showing differential pulse voltammetry (DPV) measurements of GPC-1 antigen in PBS solution; and (B) a calibration curve of DPV measurements for GPC-1 antigen in PBS solution (n=3).

FIGS. 4(A-B) illustrate (A) a plot showing differential pulse voltammetry (DPV) measurements of GPC-1 antigen in serum; (B) a calibration curve of DPV measurements for GPC-1 antigen in serum (n=3).

FIG. 5 illustrates a plot showing the results of an interference test of GPC-1 biosensor conducted by HE4 antigen.

### DETAILED DESCRIPTION

Unless specifically addressed herein, all terms used have the same meaning as would be understood by those of skilled in the art of the subject matter of the application. The following definitions will provide clarity with respect to the terms used in the specification and claims.

As used herein, the term “monitoring” refers to the use of results generated from datasets to provide useful information about an individual or an individual's health or disease status. “Monitoring” can include, for example, determination of prognosis, risk-stratification, selection of drug therapy, assessment of ongoing drug therapy, determination of effectiveness of treatment, prediction of outcomes, determination of response to therapy, diagnosis of a disease or disease complication, following of progression of a disease or providing any information relating to a patient's health status over time, selecting patients most likely to benefit from experimental therapies with known molecular mechanisms of action, selecting patients most likely to benefit from approved drugs with known molecular mechanisms where that mechanism may be important in a small subset of a disease for which the medication may not have a label, screening a patient population to help decide on a more invasive/expensive test, for example, a cascade of tests from a non-invasive blood test to a more invasive option such as biopsy, or testing to assess side effects of drugs used to treat another indication.

As used herein, the term “quantitative data” or “quantitative level” or “quantitative amount” refers to data, levels, or amounts associated with any dataset components (e.g., markers, clinical indicia,) that can be assigned a numerical value.

As used herein, the term “subject” refers to a human or another mammal. Typically, the terms “subject” and “patient” are used herein interchangeably in reference to a human individual.

As used herein, the term “bodily sample” refers to a sample that may be obtained from a subject (e.g., a human) or from components (e.g., tissues) of a subject. The sample may be of any biological tissue or fluid with, which analytes described herein may be assayed. Frequently, the sample will be a “clinical sample”, i.e., a sample derived from a patient. Such samples include, but are not limited to, bodily fluids, e.g., saliva, breath, urine, blood, plasma, or sera; and archival samples with known diagnosis, treatment and/or outcome history. The term biological sample also encompasses any material derived by processing the bodily sample. Processing of the bodily sample may involve one or more of, filtration, distillation, extraction, concentration, inactivation of interfering components, addition of reagents, and the like.

As used herein, the terms “control” or “control sample” refer to one or more biological samples isolated from an individual or group of individuals that are normal (i.e., healthy). The term “control”, “control value” or “control sample” can also refer to the compilation of data derived from samples of one or more individuals classified as normal.

As used herein, the terms “normal” and “healthy” are used interchangeably. They refer to an individual or group of individuals who have not shown any symptoms of cancer or a neurodegenerative disorder, and have not been diagnosed with cancer or a neurodegenerative disorder. In certain embodiments, normal individuals have similar sex, age, body mass index as compared with the individual from which the sample to be tested was obtained. The term “normal” is also used herein to qualify a sample isolated from a healthy individual.

As used herein, the terms “control” or “control sample” refer to one or more biological samples isolated from an individual or group of individuals that are normal (i.e., healthy). The term “control”, “control value” or “control sample” can also refer to the compilation of data derived from samples of one or more individuals classified as normal, and/or one or more individuals diagnosed with cancer or a neurodegenerative disorder.

As used herein, the term “indicative of cancer” or “indicative of a neurodegenerative disorder”, when applied to an amount of GPC-1 in a bodily sample, refers to a level or an amount, which is diagnostic of a cancer or neurodegenerative disorder such that the level or amount is found significantly more often in subjects with the cancer or neurodegenerative disorder than in subjects without the cancer or neurodegenerative disorder (as determined using routine statistical methods setting confidence levels at a minimum of 95%). Preferably, a level or amount, which is indicative of a cancer or neurodegenerative disorder, is found in at least about 60% of subjects who have the cancer or neurodegenerative disorder and is found in less than about 10% of subjects who do not have the cancer or neurodegenerative disorder. More preferably, a level or amount, which is indicative of a cancer or neurodegenerative disorder, is found in at least about 70%, at least about 75%, at least about 80%, at least about 85%, at least about 90%, at least about 95% or more in subjects who have the cancer or neurodegenerative disorder and is found in less than about 10%, less than about 8%, less than about 5%, less than about 2.5%, or less than about 1% of subjects who do not have the cancer or neurodegenerative disorder.

Embodiments described herein relate to a detection system, method, and in vitro assay for detecting, identifying, quantifying, and/or determining the levels of Glypican-1 (GPC-1) in bodily sample as well as to a detection system, method, and in vitro assay for diagnosing, identifying, staging, and/or monitoring a cancer or a neurodegenerative disorder in a subject having or suspected of having the cancer or a neurodegenerative disorder.

In some embodiments, the system and/or method for detecting, indentifying, quantifying, and/or determining a cancer or a neurodegenerative disorder can detect, indentify, quantify, and/or determine the amount or level of GPC-1 in a sample. The system can include an electrochemical biosensor, for detecting, identifying, quantifying, and/or determining the amount or level of GPC-1 in a sample, such as blood. The system and method described herein can provide a single use, disposable, and cost-effective means for simple assessment of GPC-1 in biological samples obtained by non-invasive or minimally invasive means.

In some embodiments, the electrochemical biosensor includes a substrate, a working electrode formed on a surface of the substrate and a counter electrode formed on the surface of the substrate. A dielectric layer covers a portion of the working electrode and counter electrode and defines an aperture exposing other portions of the working electrode and counter electrode. An anti-GPC-1 antibody is functionalized or chemically functionalized to a surface of the exposed portion of the working electrode. The anti-GPC-1 antibody selectively binds to GPC-1 in a sample, and the GPC-1 once bound is detectable by measuring the current flow between the working electrode and counter electrode.

The redox solution is applied to the working electrode for determining the quantity of GPC-1 in the sample bound to the anti-GPC-1 antibody. The measuring device applies voltage potentials to the working electrode and counter electrode and measures the current flow between the working electrode and counter electrode to determine the level of the GPC-1 in a bodily sample, such as a blood.

The bio-recognition mechanism of this sensor is based on the influence of the redox coupling reaction of the redox solution, such as a potassium ferrocyanide/potassium ferricyanide (K₃Fe(CN)₆/K₄Fe(CN)₆) solution, by GPC-1 and its receptor (anti-GPC-1 antibody). In the detection of GPC-1, the anti-GPC-1 antibody is used to provide a lock-and-key bio-recognition mechanism. The GPC-1 interacts with the anti-GPC-1 antibody affecting the electron charge transfer and can influence a redox coupling reaction in the redox solution applied to the working electrode. The level of GPC-1 bound to the anti-GPC-1 antibody can be determined by measuring current flow between the working and counter electrode to which the sample and redox solution has been applied and comparing the measured current to control value, which can be based on a measured current between the working electrode and counter electrode that is free of bound anti-GPC-1 antibody.

Differential pulse voltammetry (DPV) can employed as the transduction mechanism of this biosensor to determine the level of bound GPC-1. DPV applies a linear sweep voltammetry with a series of regular voltage pulses superimposed on the linear potential sweep. The current can then measured immediately before each potential change. Thus, the effect of the charging current could be minimized, achieving a higher sensitivity.

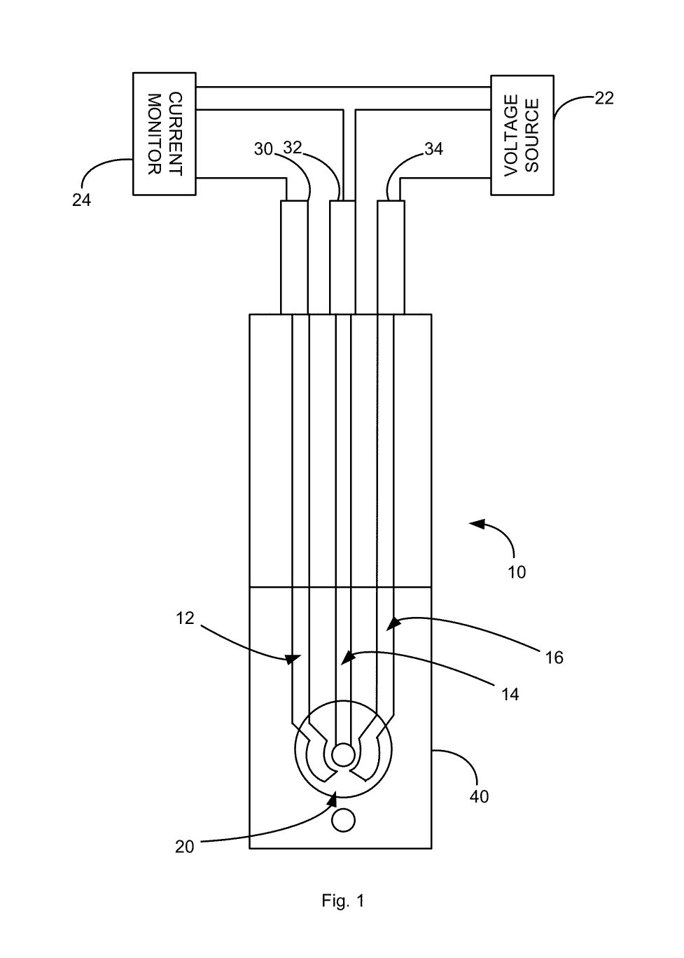

FIG. 1 illustrates a biosensor 10 of the system in accordance with an embodiment of the application. The sensor 10 is a three-electrode sensor including a counter electrode 12, a working electrode 14, and a reference electrode 16 that are formed on a surface of a substrate. A dielectric layer 40 covers a portion of the working electrode 12, counter electrode 14 and reference electrode 16. The dielectric layer 40 includes an aperture 20 that defines a detection region of the working electrode 12, counter electrode 14, and reference electrode 16, which is exposed to samples containing GPC-1 to be detected. An anti-GPC-1 antibody can be functionalized or chemically functionalized to the working electrode. The anti-GPC-1 antibody can bind selectively to GPC-1 in the biological sample.

The system further includes a measuring device that includes a voltage source 22 for applying a voltage potential to the working electrode, counter electrode, and/or reference electrode and a current monitor 24 for measuring the current flow between the working electrode and counter electrode.

The interaction of the anti-GPC-1 antibody and GPC-1 in the presence of a redox solution can be detected using electrochemical analytical techniques, such as cyclic voltammetry (CV), differential pulse voltammetry (DPV), to determine the presence of the analyte in the sample. The working electrode 14 is poised at an appropriate electrochemical potential such that the current that flows through the electrode changes when the anti-GPC-1 antibody binds to GPC-1 in the sample in the presence of the redox solution. The function of the counter electrode 12 is to complete the circuit, allowing charge to flow through the sensor 10.

The working electrode 14 and the counter electrode 12 are preferably formed of the same material, although this is not a requirement. Examples of materials that can be used for the working electrode 14 and counter electrode 12 include, but are not limited to, gold, platinum, palladium, silver, carbon, alloys thereof, and composites thereof.

The anti-GPC-1 antibody, which is functionalized or chemically functionalized to the working electrode, can be an antibody that binds selectively to GPC-1. An antibody that binds selectively to GPC-1 can be a monoclonal or polyclonal anti-GPC-1 antibody that binds selectively or specifically to GPC-1. An anti-GPC-1 antibody having binding affinities in the picomolar to micromolar range are suitable. Such interaction can be reversible or irreversible.

The term “functionalized” or “chemically functionalized,” as used herein, means addition of functional groups onto the surface of a material by chemical reaction(s). As will be readily appreciated by a person skilled in the art, functionalization can be employed for surface modification of materials in order to achieve desired surface properties, such as biocompatibility, wettability, and so on. Similarly, the term “biofunctionalization,” “biofunctionalized,” or the like, as used herein, means modification of the surface of a material so that it has desired biological function, which will he readily appreciated by a person of skill in the related art, such as bioengineering.

The anti-GPC-1 antibody may be functionalized to the working electrode covalently or non-covalently. Covalent attachment of an anti-GPC-1 antibody to the working electrode may be direct or indirect (e.g., through a linker). Anti-GPC-1 antibody may be immobilized on the working electrode using a linker. The linker can be a linker that can be used to link a variety of entities.

In some embodiments, the linker may be a homo-bifunctional linker or a hetero-bifunctional linker, depending upon the nature of the molecules to be conjugated. Homo-bifunctional linkers have two identical reactive groups. Hetero-bifunctional linkers have two different reactive groups. Various types of commercially available linkers are reactive with one or more of the following groups: primary amines, secondary amines, sulphydryls, carboxyls, carbonyls and carbohydrates. Examples of amine-specific linkers are N-hydroxysuccinimide (NHS), bis(sulfosuccinimidyl) suberate, bis[2-(succinimidooxycarbonyloxy)ethyl]sulfone, disuccinimidyl suberate, disuccinimidyl tartarate, N-succinimidyl S-acetylthioacetate, dimethyl adipimate 2HCl, dimethyl pimelimidate 2HCl, dimethyl suberimidate HCl, ethylene glycolbis-[succinimidyl-[succinate]], dithiolbis(succinimidyl propionate), and 3,3′-dithiobis(sulfosuccinimidylpropionate). Linkers reactive with sulfhydryl groups include bismaleimidohexane, 1,4-di-[3′-(2′-pyridyldithio)-propionamido)]butane, 1-[p-azidosalicylamido]-4-[iodoacetamido]butane, and N-[4-(p-azidosalicylamido)butyl]-3′-[2′-pyridyldithio]propionamide. Linkers preferentially reactive with carbohydrates include azidobenzoyl hydrazine. Linkers preferentially reactive with carboxyl groups include 4-[p-azidosalicylamido]butylamine.

Heterobifunctional linkers that react with amines and sulfhydryls include N-succinimidyl-3-[2-pyridyldithio]propionate, succinimidyl[4-iodoacetyl]aminobenzoate, succinimidyl 4-[N-maleimidomethyl]cyclohexane-1-carboxylate, m-maleimidobenzoyl-N-hydroxysuccinimide ester, sulfosuccinimidyl 6-[13-[2-pyridyldithio]propionamido]hexanoate, and sulfosuccinimidyl 4-[N-maleimidomethyl]cyclohexane-1-carboxylate. Heterobifunctional linkers that react with carboxyl and amine groups include 1-ethyl-3-[3-dimethylaminopropyl]-carbodiimide hydrochloride. Heterobifunctional linkers that react with carbohydrates and sulfhydryls include 4-[N-maleimidomethyl]-cyclohexane-1-carboxylhydrazide HCl, 4-(4-N-maleimidophenyl)-butyric acid hydrazide 2HCl, and 3-[2-pyridyldithio]propionyl hydrazide.

Alternatively, anti-GPC-1 antibodies may be non-covalently coated onto the working electrode. Non-covalent deposition of the anti-GPC-1 antibody to the working electrode may involve the use of a polymer matrix. The polymer may be naturally occurring or non-naturally occurring and may be of any type including but not limited to nucleic acid (e.g., DNA, RNA, PNA, LNA, and the like, or mimics, derivatives, or combinations thereof), amino acids (e.g., peptides, proteins (native or denatured), and the like, or mimics, derivatives, or combinations thereof, lipids, polysaccharides, and functionalized block copolymers. The anti-GPC-1 antibody may be adsorbed onto and/or entrapped within the polymer matrix.

Alternatively, the anti-GPC-1 antibody may be covalently conjugated or crosslinked to the polymer (e.g., it may be “grafted” onto a functionalized polymer).

An example of a suitable peptide polymer is poly-lysine (e.g., poly-L-lysine). Examples of other polymers include block copolymers that comprise polyethylene glycol (PEG), polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes, alkyl cellulose, hydroxyalkyl celluloses, cellulose ethers, cellulose esters, nitrocelluloses, polymers of acrylic and methacrylic esters, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, polyvinyl chloride, polystyrene, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate), poly(lactide-glycolide), copolyoxalates, polycaprolactones, polyesteramides, polyorthoesters, polyhydroxybutyric acid, polyanhydrides, poly(styrene-b-isobutylene-b-styrene) (SIBS) block copolymer, ethylene vinyl acetate, poly(meth)acrylic acid, polymers of lactic acid and glycolic acid, polyanhydrides, poly(ortho)esters, polyurethanes, poly(butic acid), poly(valeric acid), and poly(lactide-cocaprolactone), and natural polymers such as alginate and other polysaccharides including dextran and cellulose, collagen, albumin and other hydrophilic proteins, and other prolamines and hydrophobic proteins, copolymers and mixtures thereof, and chemical derivatives thereof including substitutions and/or additions of chemical groups, for example, alkyl, alkylene, hydroxylations, oxidations, and other modifications routinely made by those skilled in the art.

In one particular embodiment, the working electrode can comprise a gold working electrode that is coated with a self-assembled monolayer (SAM) of 3-mercaptopropionic acid (MPA). The MPA molecule includes a thiol functional group at one end with an affinity for gold and a carboxylic group at the other end, which can covalently bond to proteins through a peptide bond after activation. The SAM of MPT can be activated by reaction with N-(3-dimethylaminopropyl)-N′-ethylcarbodiimide hydrochloride (EDC) and N-hydroxysuccinimide (NHS), which can further react with amine groups of proteins and antibodies.

In some embodiments, the anti-GPC-1 antibody can include monoclonal and polyclonal antibodies, immunologically active fragments (e.g., Fab or (Fab)2 fragments), antibody heavy chains, humanized antibodies, antibody light chains, and chimeric antibodies. Anti-GPC-1 antibody, including monoclonal and polyclonal antibodies, fragments and chimeras, may be prepared using methods known in the art (see, for example, R. G. Mage and E. Lamoyi, in “Monoclonal Antibody Production Techniques and Applications”, 1987, Marcel Dekker, Inc.: New York, pp. 79-97; G. Kohler and C. Milstein, Nature, 1975, 256: 495-497; D. Kozbor et al., J. Immunol. Methods, 1985, 81: 31-42; and R. J. Cote et al., Proc. Natl. Acad. Sci. 1983, 80: 2026-203; R. A. Lerner, Nature, 1982, 299: 593-596; A. C. Nairn et al., Nature, 1982, 299: 734-736; A. J. Czernik et al., Methods Enzymol. 1991, 201: 264-283; A. J. Czernik et al., Neuromethods: Regulatory Protein Modification: Techniques & Protocols, 1997, 30: 219-250; A. J. Czemik et al., NeuroNeuroprotocols, 1995, 6: 56-61; H. Zhang et al., J. Biol. Chem. 2002, 277: 39379-39387; S. L. Morrison et al., Proc. Natl. Acad. Sci., 1984, 81: 6851-6855; M. S. Neuberger et al., Nature, 1984, 312: 604-608; S. Takeda et al., Nature, 1985, 314: 452-454). Antibodies to be used in the biosensor can be purified by methods well known in the art (see, for example, S. A. Minden, “Monoclonal Antibody Purification”, 1996, IBC Biomedical Library Series: Southbridge, Mass.). For example, anti-GPC-1 antibodies can be affinity purified by passage over a column to which a protein marker or fragment thereof is bound. The bound antibodies can then be eluted from the column using a buffer with a high salt concentration.

Instead of being prepared, anti-GPC-1 antibodies to be used in the methods described herein may be obtained from scientific or commercial sources.

In order to minimize any non-specific binding on the working electrode surface and blocking any open surface area of the working electrode at least one blocking agent can be applied to the surface of the working electrode once the anti-GPC-1 antibody has been functionalized or chemically functionalized to the working electrode. The blocking agent can enhance the reproducibility and sensitivity of the biosensor by minimizing non-specific interactions on the working electrode. In some embodiments, the blocking agent can include dithiothreitol or casein. The blocking agent can be applied to the surface of the working at an amount effective to minimize non-specific binding of proteins or other molecules on the surface of the working electrode.

The redox solution is applied to the working electrode for determining the quantity of GPC-1 in the sample bound to the anti-GPC-1 antibody. The redox coupling solution can include a redox mediator, such as potassium ferrocyanide/potassium ferricyanide (K₃Fe(CN)₆/K₄Fe(CN)₆), that is provided at equimolar concentration in a PBS solution.

The voltage source 22 can apply a voltage potential to the working electrode 14 and reference and/or counter electrode 16, 12, depending on the design of the sensor 10. The current between the working electrode 14 and counter electrode 16 can be measured with the measuring device or meter 24. Such current is dependent on interaction of GPC-1 in the sample with the anti-GPC-1 antibody on the working electrode.

The amount or level of current measured is proportional to the level or amount of GPC-1 in the sample. In some embodiments, where the sample is blood, once the current level generated by the sample and redox solution tested with the sensor is determined, the level can be compared to a predetermined value or control value to provide information for monitoring the presence or absence of GPC-1 in the blood sample.

In other embodiments, where the sample is a bodily sample obtained from a subject, once the current level generated by the reaction solution tested with the sensor is determined, the level can be compared to a predetermined value or control value to provide information for diagnosing or monitoring of the condition, pathology, or disorder in a subject that is associated with presence or absence of GPC-1, such as cancer or a neurodegenerative disorder.

The current level generated by sample obtained from the subject can be compared to a current level of a sample previously obtained from the subject, such as prior to administration of a therapeutic. Accordingly, the methods described herein can be used to measure the efficacy of a therapeutic regimen for the treatment of a condition, pathology, or disorder associated with the level of the GPC-1 in a subject by comparing the current level obtained before and after a therapeutic regimen. Additionally, the methods described herein can be used to measure the progression of a condition, pathology, or disorder associated with the presence or absence of the GPC-1 in a subject by comparing the current level in a bodily sample obtained over a given time period, such as days, weeks, months, or years.

The current level generated by a sample obtained from a subject may also be compared to a predetermined value or control value to provide information for determining the severity or aggressiveness of a condition, pathology, or disorder associated with GPC-1 levels in the subject. A predetermined value or control value can be based upon the current level in comparable samples obtained from a healthy or normal subject or the general population or from a select population of control subjects.

The predetermined value can take a variety of forms. The predetermined value can be a single cut-off value, such as a median or mean. The predetermined value can be established based upon comparative groups such as where the current level in one defined group is double the current level in another defined group. The predetermined value can be a range, for example, where the general subject population is divided equally (or unequally) into groups, or into quadrants, the lowest quadrant being subjects with the lowest current level, the highest quadrant being individuals with the highest current level. In an exemplary embodiment, two cutoff values are selected to minimize the rate of false positive and negative results.

The biosensor illustrated in FIG. 1 can be fabricated on a substrate 100 formed from polyester or other electrically non-conductive material, such as other polymeric materials, alumina (Al₂O₃), ceramic based materials, glass or a semi-conductive substrate, such as silicon, silicon oxide and other covered substrates. Multiple sensor devices can thus be formed on a common substrate. As will be appreciated, variations in the geometry and size of the electrodes are contemplated.

The biosensor can be made using a thin film, thick film, and/or ink-jet printing technique, especially for the deposition of multiple electrodes on a substrate. The thin film process can include physical or chemical vapor deposition. Electrochemical sensors and thick film techniques for their fabrication are discussed in U.S. Pat. No. 4,571,292 to C. C. Liu et al., U.S. Pat. No. 4,655,880 to C. C. Liu, and co-pending application U.S. Ser. No. 09/466,865, which are incorporated by reference in their entirety.

In some embodiments, the working electrode, counter electrode, and reference electrode may be formed using laser ablation, a process which can produce elements with features that are less than one-thousandth of an inch. Laser ablation enables the precise definition of the working electrode, counter electrode, and reference electrode as well as electrical connecting leads and other features, which is required to reduce coefficient of variation and provide accurate measurements. Metalized films, such as Au, Pd, and Pt or any metal having similar electrochemical properties, that can be sputtered or coated on plastic substrates, such as PET or polycarbonate, or other dielectric material, can be irradiated using laser ablation to provide these features.

In one example, a gold film with a thickness of about 300 A to about 2000 A can be deposited by a sputtering technique resulting in very uniform layer that can be laser ablated to form the working and counter electrodes. The counter electrode can use other materials. However, for the simplicity of fabrication, using identical material for both working and counter electrodes will simplify the fabrication process providing the feasibility of producing both electrodes in a single processing step. An Ag/AgCl reference electrode, the insulation layer, and the electrical connecting parts can then be printed using thick-film screen printing techniques.

The working electrode surface can then be cross-linked or biotinylated chemically in order to allow the attachment of an anti-GPC-1 antibody. The crosslinking step can be accomplished by generating thiol bonds. This can be chemically accomplished using, for example, a self-assembled monolayer (SAM) of 3-mercaptopropionic acid (MPA). The MPA molecule includes a thiol functional group at one end with an affinity for gold and a carboxylic group at the other end, which can covalently bond to proteins through peptide bond after activation. The SAM of MPT can be activated for binding to a protein, such as an antibody, by reaction with N-(3-dimethylaminopropyl)-N′-ethylcarbodiimide hydrochloride (EDC) and N-hydroxysuccinimide (NHS) that can further react with amine groups of proteins and antibodies. Similar chemical methods can be used to produce semi-stable amine-ester groups to enhance the cross linking between the antibodies and the thiol groups. Other cross-linking agent, such as 3,3′-dithiobis[sulfosuccinimidylpropionate] (DTSSP), can also be used in this process.

Biotinylation is rapid, specific and is normally unperturb to the natural function of the molecule due to the relatively small size of biotin. Streptavidin and similar chemicals such as avidin can be immobilized on the working electrode surface for a biosensor for the detection of an interaction of anti-GPC-1 antibody and GPC-1.

Following addition of an anti-GPC-1 antibody to the working electrode, the working electrode surface can be blocked using a blocking agent to minimize any non-specific molecule (e.g., protein) bonding on the electrode surface. This step will enhance the reproducibility and sensitivity of the biosensor. In some embodiments, DTT (Dithiothreitol), casein, and/or other blocking agents can be used to cover the open surface area of the working electrode and minimize any non-specific protein coverage.

In other embodiments, a plurality of biosensors can be provided on a surface of a substrate to provide a biosensor array. The biosensor array can be configured to GPC-1 concentration changes in a host of chemical and/or biological processes occurring in proximity to the array. The biosensor array can include a plurality biosensors arranged in a plurality of rows and a plurality of columns. Each biosensor can use a working electrode, a counter electrode, and a dielectric layer covering a portion of the working electrode and counter electrode and defining an aperture exposing other portions of the working electrode and counter electrode. Anti-GPC-1 antibodies for GPC-1 can be functionalized or chemically functionalized to the working electrode. The anti-GPC-1 antibodies can be the same or different for each biosensor of the array and can bind selectively to GPC-1. The biosensors of the array can be configured to provide at least one output signal representing the presence and/or concentration of GPC-1 proximate to a surface of the array. For each column of the plurality of columns or for each row of the plurality of rows, the array further comprises column or row circuitry configured to provide voltage potentials to respective biosensors in the column or row. Each biosensor in the row or column can potentially detect a different analyte and/or biased to detect different analytes.

### Example

This example describes a single-use, disposable in vitro biosensor for the detection of GPC-1. This GPC-1 biosensor was manufactured by using a cost-effective roll-to-roll process to produce a single-use, disposable device that was relatively inexpensive. This biosensor required a small quantity, (e.g., 15-20 μL), of serum or a blood test sample, without using cerebrospinal fluid (CSF), which involved an elaborate collection procedure.

The bio-recognition mechanism of this GPC-1 biosensor was based on the interaction between the antibody and the antigen of GPC-1 and its effect on the redox coupling reaction of Fe⁺²/Fe⁺³. The transduction mechanism of this biosensor employed the electrochemical analytical method, differential pulse voltammetry (DPV). DPV applied a series of regular potential pulses superimposed on the potential stair steps. The current was then measured immediately prior to each potential change. Consequently, the charging current was minimized, resulting in a higher sensitivity. In the process of establishing the bio-recognition mechanism, the antibody of GPC-1 was immobilized to the gold surface of the biosensor through a carboxylic group linking step. This immobilization process was accomplished prior to actual detection of the antigen of GPC-1. Thus, the detection time for measuring the antigen of GPC-1 was less than 1 minutes with an incubation time of 50 minutes. The total cost of preparing this GPC-1 biosensor, including the biosensor prototype, the antibody and the immobilization chemicals, was estimated to be US $3 per biosensor ensuring it was practical for a single-use, disposable in vitro biosensor for GPC-1 detection.

In this Example, the design and fabrication of the biosensor prototype, the steps in the immobilization and functionalization of the thiol group to link the antibody of GPC-1, as well as the testing of the antigen concentration of GPC-1 in phosphate buffered saline (PBS) and the undiluted human serum were described, demonstrating the effectiveness of this simple GPC-1 detection biosensor system. Interference by other antigens was investigated. HE4 antigen was employed, and this GPC-1 biosensor demonstrated excellent selectivity and specificity.

### Materials and Methods

### Regents and Apparatus

Antibody GPC-1 produced from rabbit (Cat. #HPA030571) and antigen of GPC-1 (Cat. #APREST 78918) were obtained from Sigma Aldrich (St. Louis, Mo.). Phosphate-buffered saline (PBS) 1.0 M (pH 7.4), human serum, 3-Mercaptopropionic acid (MPA), dithiothreitol (DTT), N-(3 dimethylaminopropyl)-N′-ethylcarbodiimide hydrochloride (EDC) and N-hydroxysuccinimide (NHS) were also purchased from Sigma-Aldrich (St. Louis, Mo.). Potassium hydroxide pellets, concentrated H₂SO₄ (95.0 to 98.0 w/w %) and concentrated HNO₃ (70% w/w %) were received from Fisher Scientific (Pittsburgh, Pa.). Recombinant human HE4 protein (Cat. #ab184603) was obtained from Abcam (Cambridge, Mass.). All chemicals were used without further purification. A CHI 660C (CH Instrument, Inc., Austin, Tex.) Electrochemical Workstation was used for DPV and EIS investigations. All the experiments were conducted at room temperature.

### Design of the Biosensor

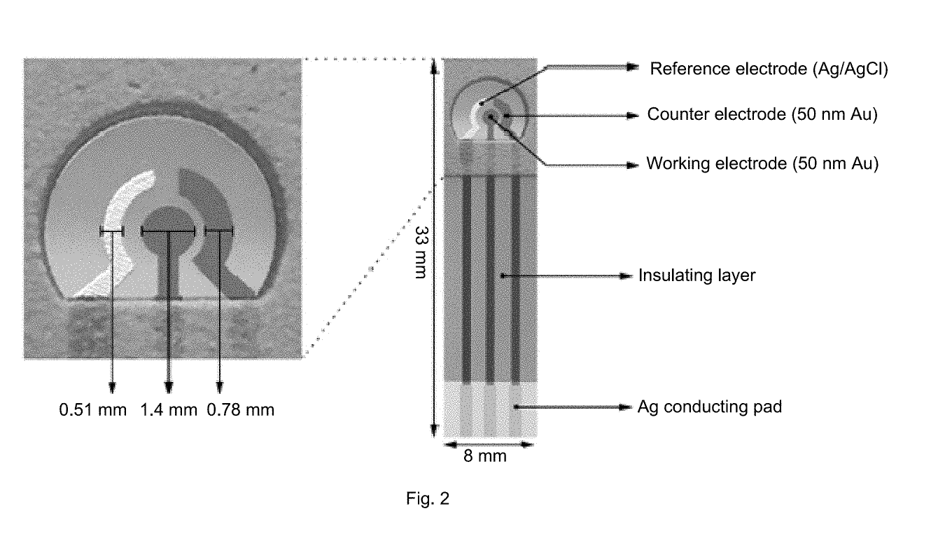

The biosensor was a three-electrode configuration electrochemical system. Both working and counter electrodes were thin gold film deposited by sputtering physical vapor deposition at an atomic level. Thus, the electrode elements were very uniform and reproducible. The thickness of the gold film was 50 nm. This biosensor prototypes were manufactured at an industrial scale on a roll-to-roll process resulting in a very cost-effective, single-use, disposable in vitro biosensor system. The reference electrode was a thick film printed Ag/AgCl electrode. Thin polyethylene terephthalate (PET) served as the substrate. Thick film silicone-free dielectric ink, Nazdar APL 34, was used to produce the insulation layer, and all the electrode connections were thick film printed silver lines. In addition to thick film printing technology, laser ablation was used to produce the dimensions and size of the biosensor. The overall dimensions of an individual biosensor are 33.0×8.0 mm². The working electrode area was 1.54 mm², accommodating 15-25 μL of liquid test sample. FIG. 2 shows the configuration of this prototype.

The bio-recognition mechanism was based on the antibody and antigen of GPC-1 interaction and its effect on a redox couple probe of Fe⁺²/Fe⁺³ Specifically, a mixed solution of K₄Fe(CN)₆ and K₃Fe(CN)₆ was used in this study to measure the conductivity on the biosensor surface. The transduction mechanism of this biosensor system was differential pulse voltammetry (DPV), which was used to measure the current outputs of different concentrations of GPC-1 antigen.

### Chemical Pretreatment of the Biosensor

The purpose of this chemical pretreatment step was to remove any minor oxide or residue on the gold film of the biosensor during the fabrication process. This step intended to enhance the reproducibility of the biosensor and the process was established based on previous reports as well as from our own studies. The sputtered gold film working and counter electrodes were relatively thin, 50 nm in thickness, and in order to maintain the integrity of the biosensor prototype, the pretreatment procedure was modified compared to treating bulk gold nano-particles or others. This cleaning process would decrease the electrode charge transfer resistance, improving the sensitivity of the biosensor. In a typical pretreatment procedure, a row of 8 or 10 biosensors was immersed in a 2M KOH solution for 10 min. After rinsing with a copious amount of de-ionized (DI) water, the biosensors were placed in a 0.05 M H₂SO₄ solution (95.0 to 98.0 w/w %) for another 10 min DI water was then used to rinse the biosensor prototypes. The biosensors were then placed in a 0.05 M HNO₃ solution (70% w/w %) for another 10 minutes. The biosensors were rinsed once more by DI water and then dried with a gently flow of nitrogen gas. Electrochemical impedance spectroscopy (EIS) was used to assess the reproducibility of these treated biosensors. The chemical pretreatment results of this GPC-1 biosensor were excellent.

Immobilization of Antibody of GPC-1 onto the Gold Film Electrodes of the Biosensor

The immobilization of the antibody of Glypican-1 (GPC-1) involved a two-step process. A thiol group was first applied providing a linkage between antibody of GPC-1 and the gold electrode surface. Self-assembled monolayers of 3-Mercaptopropionic acid (3-MPA) were used. 3-MPA molecule consisting of a thiol functional group at one end provided an excellent affinity to gold, and a carboxylic group at another end was suitable for bonding covalently to proteins through peptide amino bond after an activation procedure. However, it was important to recognize that incomplete coverage by the thiol group over the gold electrodes or any pinholes on the 3-MPA film would contribute adversely to the measurement of the biomarker, antigen of GPC-1 in this case. Dithiothreitol (DTT) was recognized in providing additional coverage and minimizing pinholes on the thiol group monolayer. Thus, 3-MPA and DTT were applied simultaneously, providing a thiol linkage and minimizing any pinhole in the coverage of the monolayer surface. Typically, 1 mM MPA and 1 mM DTT were mixed in a 15 mL pure ethanol solution and a batch of 8-10 chemically pretreated biosensors were then immersed in the solution for 24 hours. The biosensors were then rinsed with DI water. The biosensors were then functionalized by incubating in 0.1 M PBS (pH=7.4) containing 0.25M N-(3-dimethylaminopropyl)-N′-ethylcarbodiimide hydrochloride (EDC) and 0.05 M N-hydroxysuccinimide (NHS) for 5 hr at room temperature. The biosensors were then rinsed with DI water and dried by nitrogen gas again. Then 16 μL of antibody of GPC-1 solution with concentration at 7.5 μg/mL was placed onto the gold electrode surface and was incubated overnight at 4° C. The GPC-1 antibody immobilized biosensors were then rinsed with 0.1 M PBS and immersed in 1 mM BSA solution for 30 min at room temperature, covering any unbounded ester sites produced by MPA/EDC reaction. The biosensors were then rinsed with 0.1M PBS, dried by nitrogen gas, and stored at 4° C. ready for use. These preparation steps of the GPC-1 biosensors were accomplished prior to actual measurement of the GPC-1 antigen concentration in the test liquid medium.

### Results

### Detection of GPC-1 in PBS Solution Using Differential Pulse Voltammetry

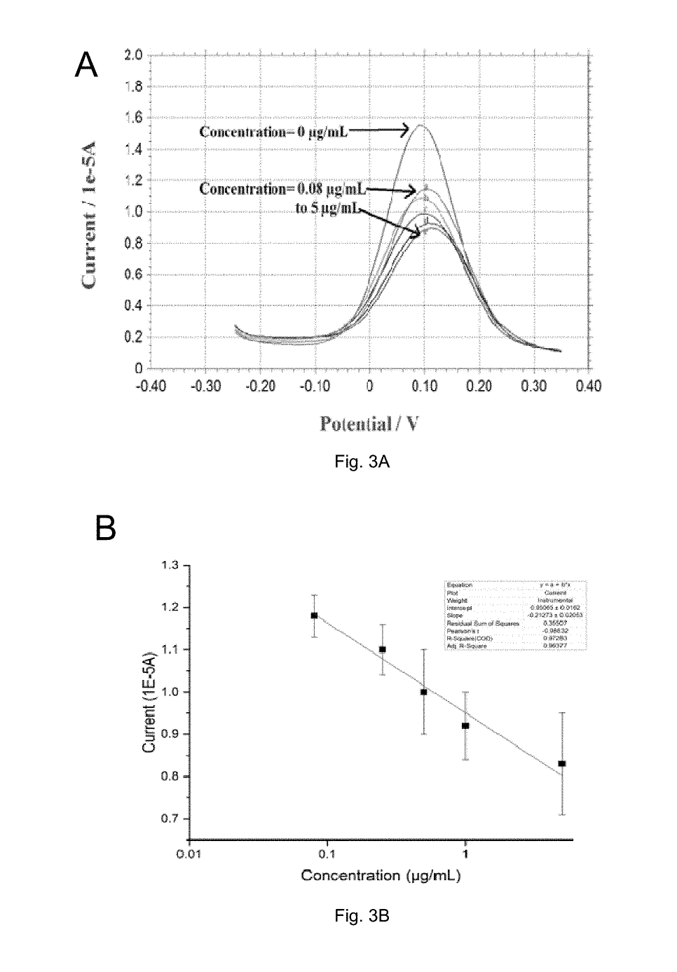

Incubation time of antigen was investigated and compared. After one-hour incubation, significant signal change was observed. Aggregation of antigen on gold may induce radicalization of protein, which may cause change of electric conductivity and lower the reproducibility. Various incubation times for the antigen of GPC-1 with minimum protein formation was empirically assessed. A 50 min. incubation time appeared to provide the most stable and reproducible signal output. This incubation time was then used throughout this study. Thus, freshly prepared GPC-1 antigen solution with a concentration range of 0.08 μg/mL to 5 μg/mL was applied to the chemically modified biosensor and incubated for 50 min. at room temperature. The biosensor was then rinsed with PBS solution and dried gently with N₂. Then, 20 μL of redox coupling solution, 5 mM of each K₄Fe(CN)₆ and K₃Fe(CN)₆, was added onto the biosensor for DPV measurement. FIGS. 3a and 3b show the DPV measurements of GPC-1 antigen concentrations of 0.08 μg/mL, 0.25 μg/mL, 0.55 μg/mL, 1.1 μg/mL, and 5 μg/mL and its calibration curve as shown in FIG. 3B with a linear equation of Y=−0.21+0.95X and an adjusted R² value of 0.96 with n=3. Higher current output indicates lower concentration of GPC-1 antigen because there is less hindrance of the electric conductivity on the biosensor surface by the interaction of the antibody and antigen of GPC-1. The highest peak in FIG. 3A shows GPC-1 antigen concentration of 0 μg/mL, which was the base line of the DPV test. Different GPC-1 antigen concentration provided a current output below the concentration=0 baseline as demonstrated in FIG. 2A.

### Detection of GPC-1 in Human Serum Using Differential Pulse Voltammetry

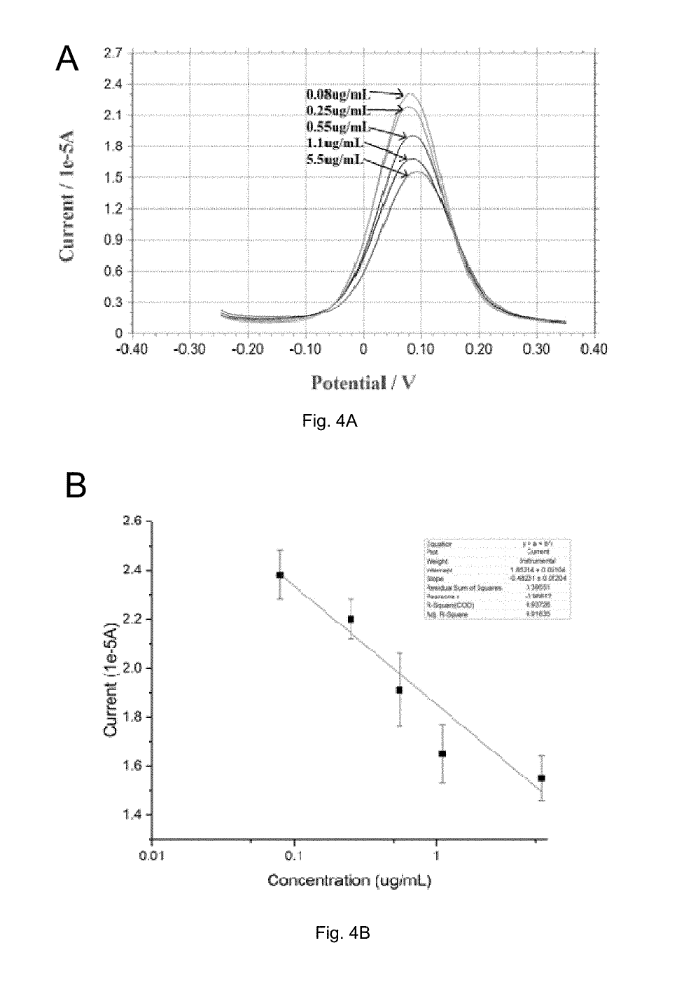

GPC-1 antigen solution was prepared in human serum with a concentration range from 0.08 μg/mL to 5 μg/mL and an incubation of 50 min was used. Similar to the test procedure used in 3.1, the antigen of GPC-1 was placed onto the biosensor and incubated for 50 min. The biosensor was rinsed by PBS solution and dried by N₂. 20 μL of the redox coupling solution, 5 mM each of K₄Fe(CN)₆ and K₃Fe(CN)₆ was placed onto the biosensor and then the DPV measurement was made. The current output of GPC-1 in serum was higher than those in the PBS test due to the higher conductivity of serum medium compared to that of PBS, and the response sequence to antigen GPC-1 concentration was consistent as expected. FIG. 4A shows the DPV measurements of GPC-1 antigen ranging from 0.08 μg/mL to 5 μg/mL and its calibration curve with a linear equation of Y=1.85-0.48X and an adjusted R² value at 0.92. FIG. 4B shows the calibration curve of the GPC-1 antigen measurement in serum with n=3.

### Interference Test of GPC-1 Biosensor Using HE4 Antigen

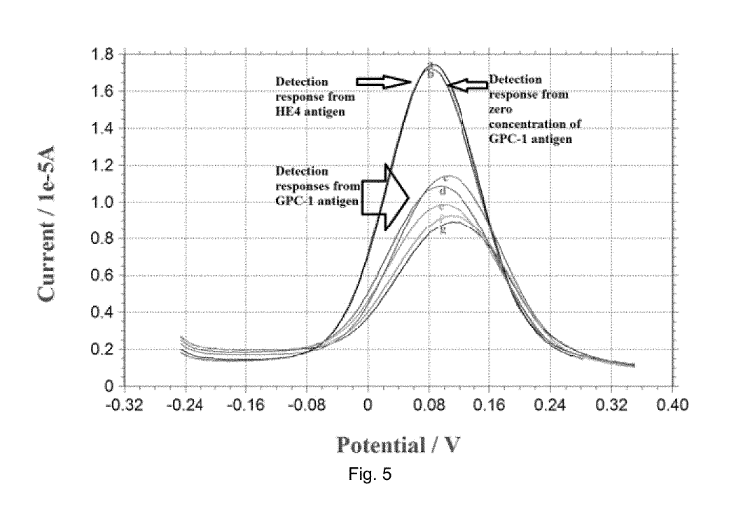

HE4 antigen was used for testing the selectivity of the GPC-1 biosensor. HE4 antigen was a general biomarker for tumor. 0.5 μg/mL HE4 antigen in PBS was used in this interference study. This quantity of HE4 antigen was placed on the chemically modified GPC-1 biosensor and incubated also for 50 min. Using the same PBS rinsing and N₂ drying procedure, and then 20 μL of redox coupling solution, 5 mM of each K₄Fe (CN)₆ and K₃Fe (CN)₆, was added onto the biosensor for DPV measurement. FIG. 5 shows that the current output of the DPV measurement of HE4 on the chemically modified GPC-1 biosensor is nearly identical to zero concentration of GPC-1 antigen, indicating that this GPC-1 biosensor was very specific and displayed no interfered by other antigens.

The results described herein show an excellent linear relationship between the biosensor current output and the GPC antigen concentration in serum over a concentration range of 0.08 μg/mL to 5 μg/mL. An interference study using HE4 antigen demonstrating that this GPC-1 biosensor was very selective and specific. The results of this example demonstrated a very practical single use, disposable in vitro GPC-1 biosensor was practical and feasible.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

Having described the invention the following is claimed:

1. A detection system for detecting Glypican-1 (GPC-1) levels in a sample, the system comprising:
a sensor that includes a substrate, a working electrode formed on a surface of the substrate; a counter electrode formed on the surface of the substrate; a dielectric layer covering a portion of the working electrode and counter electrode and defining an aperture exposing other portions of the working electrode and counter electrode; and an anti-GPC-1 functionalized or chemically functionalized to a surface of the exposed portion of the working electrode, the anti-GPC-1 antibody selectively binding to, respectively, GPC-1 in a sample and the GPC-1 once bound being detectable by measuring the current flow between the working electrode and counter electrode,
a redox solution that is applied to the working electrode for determining the quantity of GPC-1 in the sample bound, respectively, to the anti-GPC-1 antibody, and
a measuring device for applying voltage potentials to the working electrode and counter electrode and measuring the current flow between the working electrode and counter electrode.

2. The system of claim 1, wherein the working electrode and the counter electrode comprise metalized films.

3. The system of claim 2, wherein the working electrode and counter electrode independently comprise gold, platinum, palladium, silver, carbon, alloys thereof, and composites thereof.

4. The system of claim 2, wherein the metalized films are provided on the surface of the substrate by sputtering or coating the films on the surface and wherein the working electrode and the counter electrode are formed using laser ablation to define the dimensions of the working electrode and the counter electrode.

5. The system of claim 1, wherein the redox solution comprises potassium ferrocyanide/potassium ferricyanide solution.

6. The system of claim 1, further comprising a reference electrode on the surface of the substrate, the dielectric covering a portion of the reference electrode.

7. The system of claim 1, the anti-GPC-1 antibody being chemically functionalized to the surface of the working electrode coated with a 3-mercaptopropionic acid (MPA) monolayer.

8. The system of claim 1, wherein the sample comprises blood or serum.

9. A detection system for detecting GPC-1 levels in a sample, the system comprising:
a sensor that includes a substrate, a working electrode formed on a surface of the substrate; a counter electrode formed on the surface of the substrate; a dielectric layer covering a portion of the working electrode and counter electrode and defining an aperture exposing other portions of the working electrode and counter electrode; and an anti-GPC-1 antibody functionalized or chemically functionalized to a surface of the exposed portion of the working electrode, the anti-GPC-1 antibody selectively binding to, respectively, GPC-1 in a sample and the GPC-1 once bound being detectable by measuring the current flow between the working electrode and counter electrode,
an equimolar potassium ferrocyanide/potassium ferricyanide redox solution that is applied to the working electrode for determining the quantity of GPC-1 in the sample bound to, respectively, the anti-GPC-1 antibody, and
a measuring device for applying voltage potentials to the working electrode and counter electrode and measuring the current flow between the working electrode and counter electrode.

10. The system of claim 9, wherein the working electrode and the counter electrode comprise metalized films.

11. The system of claim 9, wherein the working electrode and counter electrode independently comprise gold, platinum, palladium, silver, carbon, alloys thereof, and composites thereof.

12. The system of claim 10, wherein the metalized films are provided on the surface of the substrate by sputtering or coating the films on the surface and wherein the working electrode and the counter electrode are formed using laser ablation to define the dimensions of the working electrode and the counter electrode.

13. The system of claim 9, further comprising a reference electrode on the surface of the substrate, the dielectric covering a portion of the reference electrode.

14. The system of claim 9, the anti-GPC-1 antibody being chemically functionalized to the surface of the working electrode coated with a 3-mercaptopropionic acid (MPA) monolayer.

15. The system of claim 10, wherein the sample comprises blood or serum.

16. A detection system for detecting GPC-1 levels in a sample, the system comprising:
a sensor that includes a substrate, a working electrode formed on a surface of the substrate; a counter electrode formed on the surface of the substrate; a dielectric layer covering a portion of the working electrode and counter electrode and defining an aperture exposing other portions of the working electrode and counter electrode; and an anti-GPC-1 antibody functionalized or chemically functionalized to a surface of the exposed portion of the working electrode, the anti-GPC-1 antibody selectively binding to GPC-1 in a sample and the GPC-1 once bound being detectable by measuring the current flow between the working electrode and counter electrode,
an equimolar potassium ferrocyanide/potassium ferricyanide redox solution that is applied to the working electrode for determining the quantity of GPC-1 in the sample bound to the anti-GPC-1 antibody, and
a measuring device for applying voltage potentials to the working electrode and counter electrode and measuring the current flow between the working electrode and counter electrode.

17. The system of claim 16, wherein the working electrode and the counter electrode comprise metalized films, the metalized films are provided on the surface of the substrate by sputtering or coating the films on the surface and wherein the working electrode and the counter electrode are formed using laser ablation to define the dimensions of the working electrode and the counter electrode.

18. The system of claim 16, wherein the sample comprises blood.
